(19) United States
(12) Patent Application Publication
(10) Pub. No.: US 2002/0172935 A1
(43) Pub. Date: Nov. 21, 2002

(54) Methods of screening for compounds that modulate blood vessel formation from circulating endothelial cell precursors

(76) Inventors: Christopher J. Drake, Isle of Palms, SC (US); W. Scott Argraves, Charleston, SC (US); Amanda C. LaRue, Ladson, SC (US)

Correspondence Address: NEEDLE & ROSENBERG, P.C., Suite 1200, The Candler Building, 127 Peachtree Street, N.E., Atlanta, GA, 30303-1811, US

(21) Appl. No.: 10/010,762

(22) Filed: Dec. 6, 2001

Related U.S. Application Data
(63) Continuation-in-part of application No. 09/510,687, filed on Feb. 23, 2000, now abandoned.
(60) Provisional application No. 60/251,556, filed on Dec. 6, 2000.

(30) Foreign Application Priority Data
Feb. 23, 2001 (US) PCT/US01/05661

(51) Int. Cl. C12Q 1/00; C12N 5/08

(52) U.S. Cl. 435/4; 435/366

(57) ABSTRACT

The invention provides a method of screening for agents that promote or inhibit vasculogenesis or angiogenesis. As one embodiment, the invention provides a method of screening for agents that modulate blood vessel formation from circulating endothelial cell precursors or migrating mesodermal stem cells. The invention further provides methods of using the identified agents to promote or inhibit vasculogenesis from circulating endothelial precursor cells in a tumor, tissue, organ, or graft. Also provided are methods of preventing and treating neovascular-dependent diseases using agents identified by the screening methods of the invention.

This invention was made with government support under R01HL57375-01 awarded by the Heart, Blood, and Lung Institute of the National Institutes of Health. The government has certain rights in the invention. This application claims priority to provisional U.S. patent application Ser. No. 60/251,556 which is incorporated herein in its entirety. This application is also a continuation in part of and claims priority to U.S. patent application Ser. No. 09/510,687, filed Feb. 23, 2000, which is pending and which is incorporated herein by reference in its entirety.

### BACKGROUND OF THE INVENTION

1. Field of the Invention

The present invention is related to methods of screening for agents and genes that modulate vasculogenesis from circulating endothelial cell precursors and to therapeutic uses for the identified agents. The present invention is related to the field of oncology and vascular disorders.

2. Background Art

Neovascularization refers to the growth of new blood vessels. Postnatal neovascularization has traditionally been believed to result exclusively from a process called angiogenesis, which is the proliferation, migration, and remodeling of fully differentiated endothelial cells derived from pre-existing native blood vessels. The de novo formation of blood vessels from mesodemal stem cells and endothelial cell precursors, according to traditional dogma, was thought to occur only during embryonic development by a process referred to as vasculogenesis.

Embryonic neovascularization occurs in several stages. During vasculogenesis, the most primitive stage is the appearance of endothelial precursor cells or angioblasts. These cells subsequently interact with similar cells via cell:cell adhesion molecules to form cellular “aggregates” that do not have lumens. The cells that comprise such structures are referred to as primordial endothelial cells. The first vascular structures with a lumen appear as isolated vessel segments. These segments then interconnect to form vascular networks. After the formation of the first blood vessels, additional vessels are formed by either continued vasculogenesis or by the second neovascular process, angiogenesis, the growth of vessels from preexisting vessels.

Normal neovascularization has been thought to have important roles. Specifically, vasculogenesis has been thought to play an important role in embryonic development, whereas angiogenesis has been implicated in a variety of physiological processes such as wound healing, organ regeneration and female reproductive processes such as follicle development in the corpus luteum during ovulation and placental growth with pregnancy. Folkman & Shing, 1992, J. Biological Chem. 267(16):10931-34. Uncontrolled angiogenesis, in contrast, has been associated with diseases, such as diabetes and malignant solid tumors that rely on vascularization for growth. See Folkman, 1990; Weidner et al., 1991. In diabetes, following vascular occlusion, new capillaries that invade the vitreous subsequently bleed and cause blindness. In addition, in arthritis, new blood vessels invade the joint and destroy the articular cartilage.

Because only angiogenesis was traditionally believed to have a postnatal role, treatment strategies have focused on promoting or interrupting angiogensis. Thus, treatment has been directed to the endothelial cells of existing blood vessels rather than mesodermal stem cells or endothelial cell precursors. Recent studies, in contrast to the traditional dogma, have suggested that vasculogenesis, as well as angiogenesis, may play a postnatal role. See Isner and Asahara (1999); Springer et al. (1998). Little effort has been made to date to identify the agents that affect postnatal vasculogenesis from circulating endothelial cell precursors. This is due, in part, to the fact that there has been no method of distinguishing agents that specifically affect vasculogenesis from circulating endothelial cell precursors.

### SUMMARY OF THE INVENTION

It is an object of the invention to provide a means of screening for agents and nucleic acids that specifically modulate vasculogenesis from circulating endothelial cell precursors. It is a further object of the invention to provide methods of using the identified agents and nucleic acids for therapeutic uses. It is a further object of the invention to provide a means of screening for agents and nucleic acids that specifically modulate vasculogenesis or angiogenesis. It is a further object of the invention to provide methods of using the identified agents and nucleic acids for therapeutic uses. Another object of the invention is to provide a method of identifying stem cells of unknown endothelial cell potential as cells that can differentiate into endothelial cell precursors or endothelial cells.

Thus, the invention further provides a method of screening for agents that modulate blood vessel formation from circulating endothelial precursors or migrating mesodermal stem cells. Specifically, the invention provides a method of screening for an agent that promotes vasculogenesis or inhibits vasculogenesis, comprising the steps of (a) contacting one or more embryonic vascular networks with the agent to be screened, under conditions in which extraembryonic mesodermal stem cells, or derivatives thereof, can migrate to the embryonic vascular network or networks; (b) detecting, in the vascular network or networks, endothelial cells or endothelial cell precursors derived from extraembryonic mesodermal stem cells, or derivatives thereof; and (c) comparing the endothelial cells or endothelial cell precursors derived from extraembryonic mesodermal stem cells, or derivatives thereof, in the networks contacted with the agent to be screened, with the endothelial cells or endothelial cell precursors derived from extraembryonic mesodermal stem cells, or derivatives thereof, in untreated networks, an increase in endothelial cells or endothelial cell precursors derived from extraembryonic mesodermal stem cells, or derivatives thereof, in the network or networks contacted with the agent to be screened indicating an agent that promotes vasculogenesis and a decrease in endothelial cells or endothelial cell precursors derived from extraembryonic mesodermal stem cells, or derivatives thereof, in the network or networks contacted with the agent to be screened indicating an agent that inhibits vasculogenesis.

By “circulating” or “migrating” in reference to endothelial precursors or mesodermal stem cells is meant that the cells move from a point of origin to reach, contribute to, or originate the formation of the nascent vascular network. For example, the precursor or stem cells could be attracted to the region by chemotaxis. Once the region of blood vessel formation is reached, the precursor or stem cells divide and/or differentiate to form endothelial cells that are integrated into the structure of the vascular networks and, ultimately, into the endothelial layer of the blood vessel wall.

The invention also provides a method of screening for an agent that promotes or inhibits vasculogenesis, comprising the steps of (a) co-culturing extraembryonic mesodermal stem cells and intraembryonic mesodermal stem cells, under conditions that allow formation of one or more vascular networks; (b) contacting the co-culture with the agent to be screened; (c) detecting, in one or more vascular networks, endothelial cells or endothelial cell precursors derived from extraembryonic mesodermal stem cells, or derivatives thereof, and (d) comparing the endothelial cells or endothelial cell precursors derived from extraembryonic mesodermal stem cells, or derivatives thereof, in the vascular network or networks in the culture contacted with the agent to be screened, with the endothelial cells or endothelial cell precursors derived from extraembryonic mesodermal stem cells, or derivatives thereof, in the vascular network or networks of the untreated cultures. An increase in endothelial cells or endothelial cell precursors derived from extraembryonic mesodermal stem cells, or derivatives thereof, in the vascular networks in the culture contacted with the agent to be screened indicates an agent that promotes vasculogenesis; whereas, a decrease in endothelial cells or endothelial cell precursors derived from extraembryonic mesodermal stem cells, or derivatives thereof, in the vascular networks in the culture contacted with the agent to be screened indicates an agent that inhibits vasculogenesis.

The invention further provides a method of screening for an agent that promotes or inhibits vasculogenesis, comprising the steps of culturing mesodermal stem cells; contacting the mesodermal stem cells with the agent to be screened; detecting endothelial cells or endothelial cell precursors in the culture; and comparing the endothelial cells or endothelial cell precursors in the culture to be screened, with the endothelial cells or endothelial cell precursors in a control culture, an increase in endothelial cells or endothelial cell precursors in the culture to be screened indicating an agent that promotes vasculogenesis and a decrease in endothelial cells or endothelial cell precursors in the culture to be screened indicating an agent that inhibits vasculogenesis. In a preferred embodiment the mesodermal stem cells are allantoic cells. In an alternative embodiment, embryonic stem cells can be used instead of mesodermal stem cells in the method of screening for an agent that promotes or inhibits vasculogenesis.

Also provided is a method of screening for an agent that promotes or inhibits angiogenesis, comprising the steps of culturing allantoic cells; contacting the allantoic cells with the agent to be screened; detecting endothelial cells or endothelial cell precursors in the culture; and comparing the endothelial cells or endothelial cell precursors in the culture to be screened, with the endothelial cells or endothelial cell precursors in a control culture, an increase in endothelial cells or endothelial cell precursors in the culture to be screened indicating an agent that promotes angiogenesis and a decrease in endothelial cells or endothelial cell precursors in the culture to be screened indicating an agent that inhibits angiogenesis. Once endothelial cells form in the allantoic culture, angiogenesis can occur. Thus, a culture of allantoic cells or an ex vivo culture of an allantois that includes both mesodermal stem cells and endothelial cells can be used to screen for factors that affect angiogenesis and/or vasculogenesis.

The invention provides a method of promoting or inhibiting vasculogenesis or angiogenesis in a tissue or organ, comprising contacting the tissue or organ with a therapeutically effective amount of the agent identified by the screening methods of the invention. Also provided are methods of preventing and treating neovascular-dependent diseases (for example, retinopathy, neovascularization of the cornea or iris, solid tumors, cancer, and hemangioma). Thus, the invention provides a method of preventing a neovascular-dependent disease in a subject or treating a neovascular-dependent disease in a subject, comprising administering to the subject a therapeutically effective amount of the agent identified by the screening methods of the present invention.

The present invention also provides a method of screening for an agent that stabilizes vasculature or promotes remodeling of vasculature, comprising the steps of culturing allantoic cells, under conditions that allow the formation and remodeling of vasculature; contacting the vasculature with the agent to be screened; detecting the remodeling of the vasculature; and comparing the remodeling in the culture to be screened with the remodeling in a control culture, less remodeling in the culture to be screened indicating an agent that stabilizes vasculature and more remodeling in the culture to be screened indicating an agent that promotes remodeling of vasculature.

The present invention further provides a method of screening for genes involved in promoting or inhibiting neovascularization (i.e., vasculogenesis and/or angiogenesis). The screening method comprises the steps of culturing allantoic cells in the presence or absence of an agent that promotes or inhibits differentiation of mesodermal stem cells into endothelial cells or endothelial precursor cells or promotes or inhibits the differentiation of endothelial cell precursors into endothelial cells; isolating nucleic acids from the allantoic cells; and detecting the nucleic acids present at higher or lower levels from the allantoic cells cultured in the presence of the agent as compared to the allantoic cells cultured in the absence of the agent, wherein the nucleic acid present at higher or lower levels in allantoic cells cultured in the presence the agent indicates genes involved in promoting or inhibiting neovascularization.

The invention further provides methods of using the identified nucleic acids to promote or inhibit vasculogenesis or angiogenesis in a tumor, tissue, organ, or graft. A method of preventing a neovascular-dependent disease in a subject or treating a subject with a neovascular-dependent disease is provided, comprising administering to the subject a therapeutically effective amount of either a nucleic acid that blocks expression of the gene identified by the screening method and further identified to promote neovascularization or a nucleic acid that encodes a protein that promotes expression of the gene identified by the screening method and further identified as inhibiting neovascularization. Also, provided is a method of promoting vascularization of a tissue, organ, or graft in a subject, comprising administering to the subject either a nucleic acid that blocks expression of the gene identified by the screening method and further identified to inhibit neovascularization or a nucleic acid that encodes a protein that promotes expression of the gene identified by the screening method and further identified as promoting neovascularization.

The invention further provides a method of determining whether stem cells of unknown endothelial cell potential can be promoted to differentiate into endothelial cell precursors, comprising culturing the stem cells under conditions that allow the cells to differentiate into endothelial cell precursors; and determining the presence of endothelial cell precursors by detecting the co-expression of TAL1 and FLK1.

### BRIEF DESCRIPTION OF THE DRAWINGS

FIG. 1 shows the temporal expression pattern of various vascular marker proteins during allantoic development. The plotted patterns were determined using confocal microscopic analysis of murine allantoides labeled with antibodies to the respective proteins.

FIG. 2a shows PECAM immunolabeling of a 7.5 dpc murine allantois. At 7.5 dpc, there is a lack of PECAM labeling in the allantois.

FIG. 2b shows PECAM immunolabeling of an 8.2 dpc murine allantois. By 8.2 dpc a PECAM-positive central vessel extends along the length of the allantois with the more mature portion of the vessel being found at the allantoic base (bottom).

FIG. 2c shows PECAM immunolabeling of a late 8.5 dpc murine allantois. By late 8.5 dpc, the allantois has fused with the maternal placental vasculature and has developed a dense vascular network surrounding the central vessel.

FIG. 3a shows a normal 7.0 dpc murine allantois cultured for 24 hours and immunolabeled with PECAM antibodies.

FIG. 3b shows an 7.0 dpc allantois cultured for 24 h in the presence of FLT-1 receptor (4μg/ml) and immunolabeled with PECAM antibodies. Treatment with soluble FLT- 1 receptor results in the loss of a normal polygonal vascular arrangement.

FIG. 3c shows a 7.0 dpc allantois cultured for 24 hours in the presence of VEGF (2 μg/ml) and immunolabeled with PECAM antibodies. Exposure to VEGF leads to an overall sinusoidal vascular pattern.

FIG. 3d shows a normal 8.0 dpc murine allantois cultured for 24 hours and immunolabeled with antibodies to PECAM.

FIG. 3e shows an 8.0 dpc allantois cultured for 24 hours in the presence of FLT-1 receptor (4 μg/ml) and immunolabeled with antibodies to PECAM.

FIG. 3f shows an 8.0 dpc allantois cultured 24 hours in the presence of VEGF (2 μg/ml) and immunolabeled with antibodies to PECAM.

FIG. 4 shows the results of flow cytometric analysis of the expression of vascular related proteins in 8-8.5 dpc mouse allantoides.

### DETAILED DESCRIPTION OF THE INVENTION

The present invention may be understood more readily by reference to the following detailed description of preferred embodiments of the invention and the Examples included therein.

Before the present methods are disclosed and described, it is to be understood that this invention is not limited to specific methods or to particular formulations, as such may, of course, vary. It is also to be understood that the terminology used herein is for the purpose of describing particular embodiments only and is not intended to be limiting.

As used in the specification and in the claims, “a” can mean one or more, depending upon the context in which it is used.

The invention provides a method of screening for an agent that promotes or inhibits vasculogenesis, comprising the steps of culturing mesodermal stem cells; contacting the mesodermal stem cells with the agent to be screened; detecting endothelial cells or endothelial cell precursors in the culture; and comparing the endothelial cells or endothelial cell precursors in the culture to be screened, with the endothelial cells or endothelial cell precursors in a control culture, an increase in endothelial cells or endothelial cell precursors in the culture to be screened indicating an agent that promotes vasculogenesis and a decrease in endothelial cells or endothelial cell precursors in the culture to be screened indicating an agent that inhibits vasculogenesis.

As used throughout, by “mesodermal stem cells” is meant stem cells of origin, including , for example, splanchnic mesodermal origin, that have the capacity to differentiate into cells of endothelial lineage. The mesodermal stem cell, therefore, can be a multipotent cell that can differentiate, directly or indirectly through intermediate cell types, into endothelial precursor cells or endothelial cells. The mesodermal stem cells can be derived from an embryonic or nonembryonic source. By “nonembryonic” is meant fetal or postnatal. The embryonic period is considered to be early prenatal development, and specifically, in the human, the first eight weeks following fertilization. One skilled in the art would recognize that the equivalent period in other mammalian species would constitute the embryonic period.

Preferably, the mesodermal stem cells are splanchnic mesodermal stem cells, more preferably, mammalian splanchnic mesodermal stem cells. Even more preferably, the splanchnic mesodermal stem cells are allantoic mesodermal stem cells. The allantoic mesodermal stem cell culture can comprise an ex vivo allantoic culture or aggregates of dissociated allantoic cells. The aggregates can be in the form of spheroids. Alternatively, the mesodermal stem cells can be bone marrow mesodermal stem cells, connective tissue mesodermal stem cells, or immortalized mesoderm stem cells. The cultures of bone marrow mesodermal stem cells, connective tissue mesodermal stem cells, or immortalized mesoderm stem cells can be aggregates of dissociated cells.

The mesodermal stem cells are not differentiated endothelial cells. The use of mesodermal stem cells in the screening procedures of the present invention has an advantage over the use of endothelial cells because, when endothelial cells are used for screening, only angiogenesis can be evaluated. Important aspects of de novo vessel formation by vasculogenesis are overlooked using screening methods with only endothelial cells. The allantoic mesodermal stem cells also have a particular advantage because the allantois is relatively devoid of either endodermal or ectodermal cells, and, early in development, the allantois constitutes relatively pure embryonic splanchnic mesodermal stem cells. Thus, in a preferred embodiment of the invention, the mesodermal stem cell culture is relatively devoid of either endodermal or ectodermal stem cells or both. Preferably, the mesodermal stem cell culture is relatively devoid of endothelial cells prior to contact with the agent to be screened for vasculogenic properties. This provides a distinct advantage over previous methods known in the art in which the inducing role of endodermal and ectodermal cells cannot be ruled out. By “relatively devoid of endodermal or ectodernal stem cells” is meant a mesodermal stem cell culture that contains no more than about 20%, 10%, 5%, or 1% endodermal and ectodermal stem cells. Preferably, the culture is completely devoid of endodermal and ectodermal stem cells and contains less than 0.1% endodermal and ectodermal stem cells. By “relatively devoid of endothelial cells” is meant a mesodermal stem cell culture that contains no more than about 20%, 10%, 5%, or 1% endothelial cells prior to contact with the agent to be screened. Preferably, the culture is completely devoid of endothelial cells and contains less than 0.1% endothelial cells prior to contact with the agent to be screened.

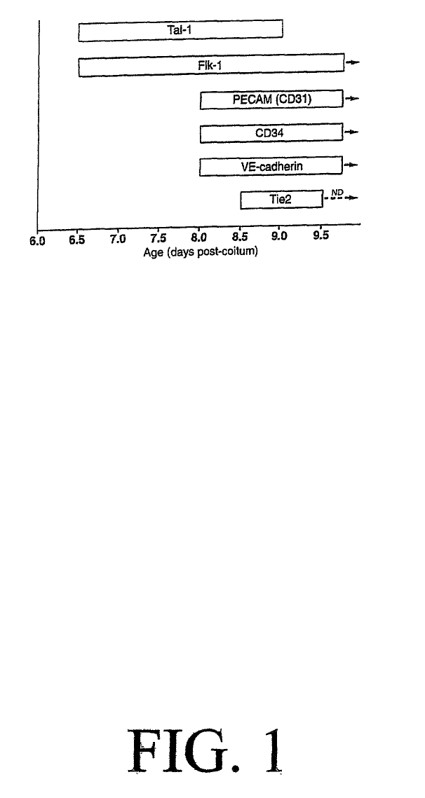

By “endothelial cells or endothelial precursor cells,” as used throughout, is meant cells that show at least one phenotypic characteristic of an endothelial cell or endothelial precursor cell. Such phenotypic characteristics can include expression of vascular marker proteins and the ability to form primitive blood vessels called vascular networks. The endothelial cells or endothelial cell precursors can be detected by one or more vascular marker proteins including, for example, TAL1, Flk1, CD34, VE-cadherin, Tie 2, and platelet/endothelial cell adhesion molecule (PECAM; also, referred to as “CD31”). The present invention provides a characterization of the time course of the appearance of these markers in vasculogenesis. See FIG. 1. Early endothelial cell precursors (angioblasts) are identifiable as cells that co-express TAL1 and Flk1. The early endothelial cell precursors are comparable to mouse allantoic endothelial cell precursors detectable between days 6.5 and 8.5 post-coitum. Furthermore, these early endothelial cell precursors do not express PECAM (CD31), CD34, VE-cadherin, and Tie2 or express these markers only at low levels. By “low levels” is meant less than 5 times the assay background level, and, more preferably, less than 2.5 times the background level, and, even more preferably, the same as background levels. Late endothelial cell precursors are comparable to mouse allantoic endothelial cell precursors detectable between days 8.5 and 9.0 post-coitum. The late endothelial cell precursors express TAL1 and Flk-1 as well as PECAM, CD34, VE-cadherin. Additionally, late endothelial cell precursors that are comparable to mouse allantoic endothelial cell precursors detectable between days 8.5 and 9.0 post-coitum also express Tie2. Endothelial cells, comparable to mouse allantoic endothelial cells detectable after day 9.0 post-coitum, express Flk-1, PECAM, CD34, VE-cadherin, but do not express TAL1, or express it only at low levels. Early endothelial cells that are comparable to mouse allantoic endothelial cells detectable between days 9.0 and 9.5 post-coitum can also express Tie2. Antibodies to the specific markers can be used to detect the presence of the markers.

A number of criteria are used to evaluate the potential alterations in vessel development and thereby identify agents that promote or inhibit neovascularization or evaluate the effectiveness of these agents. An indicator of the inhibitory effect of an agent to be screened is a failure of the culture to form vascular networks (i.e., unconnected vessel fragments) or a disruption in normal vascular network patterns. These changes can be associated with or without a concommitant decrease or increase in the number of endothelial cells and/or endothelial precursor cells. Additionally, other criteria such as angioblast and endothelial cell expression of specific proteins (i.e. TAL1, Flk 1, CD31, CD34, VE-cadherin, Tie2) in the correct temporal pattern, angioblast and endothelial cell numbers, and apoptosis can be evaluated. For example, in the mesodermal cell culture, the endothelial cells or endothelial cell precursors form vascular networks, and an increase in the number or complexity of the vascular networks in the culture to be screened indicates an agent that promotes vasculogenesis. The endothelial cells or endothelial cell precursors can be detected before vascular networks are formed or after vascular networks are formed. The morphological characteristics of the vascular networks can be assessed immunohistochemically using antibodies to the specific markers or by other techniques known in the art (e.g., in situ hybridization). The vascular networks can then be visualized using fluorescence, dark field, traditional light, or confocal microscopy.

By “an increase in the number of endothelial cells or endothelial precursor cells” is meant an increase by as little as 1%, 2%, 3%, 4%, 5%, 10%, 20%, 30%, 40%, 50%, 60%, 70%, 80%, 90%, and 100% and up to and even exceeding 200%, 300%, 400%, 500%, 600%, as well as any values in between in the actual number of cells or in the amount of an endothelial cell or endothelial precursor cell marker as compared to a control. Thus, by “promoting vaculogensis” is meant increasing the number of endothelial cells or endothelial cell precursors by any amount, including as little as 1%, 2%, 3%, 4%, 5%, 10%, 20%, 30%, 40%, 50%, 60%, 70%, 80%, 90%, and 100% and up to and even exceeding 200%, 300%, 400%, 500%, 600%, as well as any values in between.

“A decrease in endothelial cells or endothelial precursor cells” is meant a decrease by as little as 1%, 2%, 3%, 4%, 5%, 10%, 20%, 30%, 40%, 50%, 60%, 70%, 80%, 90%, and 100% , as well as any values in between, in the actual number of cells or in the amount of an endothelial cell or endothelial precursor cell marker as compared to a control. Thus, by “inhibiting vaculogensis” is meant decreasing the number of endothelial cells or endothelial cell precursors by any amount, 1%, 2%, 3%, 4%, 5%, 10%, 20%, 30%, 40%, 50%, 60%, 70%, 80%, 90%, and 100%, as well as any values in between.

It is understood that either the number of endothelial cells or endothelial cell precursors may increase or decrease without an increase or decrease in the other. For example, in the case of promoting angiogenesis, the number of endothelial cells only, without a concomitant increase in the number of endothelial cell precursors can occur. Likewise, the levels of markers or combinations of markers that indicate endothelial cells may increase with angiogenesis without an increase in markers or combinations of markers specific for endothelial cell precursors. With vasculogenesis, increases in endothelial cell precursors and markers or combinations of markers for endothelial cell precursors can occur in the presence or absence of increases in endothelial cells and markers or combinations of markers for endothelial cells. Thus, it is understood that either the amount of endothelial cell or endothelial precursor cell marker or markers may increase without an increase in the number of cells, or vice versa. Similarly, the amount of endothelial cell or endothelial precursor cell marker or markers may decrease without a decrease in the number of cells, or vice versa. For example, the synthesis of the marker or markers by each cell may increase without an increase in the total number of cells. The synthesis of the marker or markers by each cell, conversely, may decrease but the number of endothelial cells or endothelial cell precursors may increase.

By “an increase in vascular networks” is meant an increase in the number of vascular networks or an increase in the complexity of vascular networks. The complexity of a vascular network can be assessed by evaluating the branch points or the total area of the vascular network, a more complex vascular network having more branch points and/or great area. Thus, an increase in any one of these parameters can be by as little as 1%, 2%, 3%, 4%, 5%, 10%, 20%, 30%, 40%, 50%, 60%, 70%, 80%, 90%, and 100% and up to and even exceeding 200%, 300%, 400%, 500%, 600%, as well as any values in between. By “decrease in vascular networks” is meant a decrease in the number of vascular networks or a decrease in the complexity of vascular networks, in the actual number of cells, in the amount of an endothelial cell or endothelial precursor cell marker, or a disruption in the vascular pattern. It is understood that one or a combination of indicators may show a decrease. The decrease in any one of the listed parameters can be by as little as 1%, 2%, 3%, 4%, 5%, 10%, 20%, 30%, 40%, 50%, 60%, 70%, 80%, 90%, and 100%, as well as any value in between.

As used throughout, by “culturing” is meant the placement of mesodermal stem cells or mesoderm stem cell-containing tissue or organ in a medium for seconds, minutes, hours, days, weeks, or months.

As used throughout, by “contacting” is meant an instance of exposure of at least one substance (e.g., a culture, allantois, explant, organ, tissue, graft, or tumor) or cell (e.g., a mesodermal stem cell, allantoic cells, or embryonic stem cell) to an agent. The cell or substance can be contacted with an agent, for example, by adding the agent to the culture medium (by continuous infusion, by bolus delivery, or by changing the medium to a medium that contains the agent) or by adding the agent to the extracellular fluid in vivo (by local delivery, systemic delivery, intravenous injection, bolus delivery, continuous infusion or by delivery of an agent conjugated or associated with some matrix for limited release or controlled time release). The duration of “contact” with a cell, group of cells, or substance is determined by the time the agent is present at physiologically effective levels or at presumed physiologically effective levels in the medium or extracellular fluid bathing the cell. Preferably, in the screening methods of the present invention, mesodermal stem cells, allantoic cells, or embryonic stem cells are contacted with the agent to be screened for 1-48 hours and more preferably for 24 hours, but such time would vary based on the half life of the agent and could be optimized by one skilled in the art using routine experimentation.

The invention further provides a method of screening for an agent that promotes or inhibits vasculogenesis, comprising the steps of culturing embryonic stem cells, under conditions that allow formation of aggregates; contacting the aggregates with the agent to be screened; detecting endothelial cells or endothelial cell precursors in the aggregates; and comparing the endothelial cells or endothelial cell precursors in the culture to be screened, with the endothelial cells or endothelial cell precursors in a control culture, an increase in endothelial cells or endothelial cell precursors in the culture to be screened indicating an agent that promotes vasculogenesis and a decrease in endothelial cells or endothelial cell precursors in the culture to be screened indicating an agent that inhibits vasculogenesis. The aggregates can be spheroids or embryoid bodies. The endothelial cells or endothelial cell precursors can form vascular networks like the endothelial cells and endothelial cell precursors in the mesodermal stem cell cultures. Thus, the number and complexity of vascular networks can similarly be detected and assessed. Also, a disruption in normal vascular patterns can be detected and assessed.

Also provided is a method of screening for an agent that promotes or inhibits angiogenesis, comprising the steps of culturing allantoic cells; contacting the allantoic cells with the agent to be screened; detecting endothelial cells or endothelial cell precursors in the culture; and comparing the endothelial cells or endothelial cell precursors in the culture to be screened, with the endothelial cells or endothelial cell precursors in a control culture, an increase in endothelial cells or endothelial cell precursors in the culture to be screened indicating an agent that promotes angiogenesis and a decrease in endothelial cells or endothelial cell precursors in the culture to be screened indicating an agent that inhibits angiogenesis. Once endothelial cells form in the allantoic culture, angiogenesis can occur. Thus, a culture of allantoic cells or an ex vivo culture of an allantois that includes both mesodermal stem cells and endothelial cells can be used to screen for factors that affect angiogenesis and/or vasculogenesis.

As used throughout, the detecting step of the methods of the present invention comprises an assay selected from the group consisting of an immunohistological assay, an immunocytochemical assay, a flow cytometric assay, an ELISA, a radioimmunoassay, a Western blot assay, a RT-PCR, and an oligonucleotide microarray.

The invention provides a method of promoting or inhibiting vasculogenesis or angiogenesis in a tissue or organ, comprising contacting the tissue or organ with a therapeutically effective amount of the agent identified by the screening method of the invention. There are various conditions in which vasculogenesis or angiogenesis is desired, including, for example, for promoting wound and ulcer healing, organ or tissue regeneration, vascularization of a transplanted tissue or organ, or establishment of collateral circulation (e.g., following a vascular occlusion of a coronary or cerebral vessel or for treating or preventing peripheral vascular disease). The contacting step can be either in vivo, ex vivo, or in vitro. For example, a tissue (e.g., skin) or organ (e.g., pancreas, liver, heart, etc.) to be transplanted into a host can be contacted ex vivo prior to transplantation into a donor. The tissue or organ, alternatively, can be contacted in vivo prior to removal from the donor or after transplantation into the recipient. Similarly, a cellular transplant (e.g., pancreatic islet cells) can similarly be treated with an agent identified by the screening method of the invention.

There are also numerous conditions in which inhibition of vasculogenesis or angiogenesis is desired, including, for example, in a tumor or in any pathology associated with neovascularization. The invention provides a method of preventing a neovascular-dependent disease in a subject or treating a neovascular-dependent disease in a subject, comprising administering to the subject a therapeutically effective amount of the agent identified by the screening method of the present invention. As used throughout, “treating” or “preventing” means reducing or preventing any of the clinical manifestations of the neovascular-dependent disease. Thus, one skilled in the art would know how to determine the efficacy of treatment or prevention.

In general, “a therapeutically effective amount of an agent” is that amount needed to achieve the desired result or results (e.g., promoting vasculogenesis or angiogenesis or inhibiting vasculogenesis or angiogenesis). One of ordinary skill in the art will recognize that the potency and, therefore, a “therapeutically effective amount of an agent” can vary for the various agents used in this invention. One skilled in the art can readily assess the potency of a candidate agent that promotes or inhibits neovascularization. For example, potency can be determined by measuring tumor growth or wound repair; an amount that slows or prevents tumor growth would be a therapeutically effective amount of an agent that inhibits neovascularization, whereas an amount that increases the rate of wound healing would be a therapeutically effective amount of an agent that promotes neovascularization. Alternatively, vasculature can be imaged using techniques known in the art, including, for example, angiography (fluorescein angiography, radio-angiography, or indocyanine green angiography). The efficacy of an agent in preventing or treating a selected condition can be similarly evaluated by one skilled in the art.

The neovascular-dependent disease can be either a vasculogenic-dependent or angiogenic-dependent disease or can have characteristics of both. By “a vasculogenic-dependent disease” or “an angiogenic -dependent disease” is meant a disease, disorder, or condition that either does not occur or does not progress in the absence of postnatal vasculogenesis or angiogensis, respectively, or in the absence of both vasculogenesis and angiogenesis. Vasculogenic-dependent or angiogenic diseases include but are not limited to retinopathy (e.g., diabetes retinopathy, retinopathy of prematurity, sickle cell-induced retinopathy, and chronic retinal detachment), inflammatory diseases (e.g., retinal periphlebitis, sarcoidosis, Behcat's disease, posterior uveitis, chronic inflammatory diseases of the posterior segment), carotid occlusive diseases of the eye, rubeosis iridis, neovascularization of the cornea or iris, solid tumors, cancer, and hemangioma.

The agents used in this invention are administered to a subject in need thereof by commonly employed methods for administering agents in such a way to bring the agent in contact with the tumor, tissue, organ, or graft where either promotion or inhibition of neovascularization is desired. The agents of the present invention can be administered orally, parenterally, transdermally, extracorporeally, topically or the like, although oral or topical administration is typically preferred. Parenteral administration of the agents of the present invention, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, subcutaneous, intramuscular, intraperitoneal, intravenous, intra-articular and intratracheal routes. A more recently revised approach for parenteral administration involves use of a slow release or sustained release system such that a constant dosage is maintained. See, e.g., U.S. Pat. No. 3,610,795, which is incorporated by reference herein. The agents can also be administered using polymer based delivery systems, including, for example, microencapsulation as described in Langer (1998). The agents of the present invention can be administered using gene therapy methods of delivery. See, e.g., U.S. Pat. No. 5,399,346, which is incorporated by reference herein. Using a gene therapy method of delivery, primary cells transfected with the gene for the agent of the present invention can additionally be transfected with tissue specific promoters to target specific tumors, organs, tissue, or grafts.

The dosage of the agent varies depending on the type of neovascular-dependent disease, degree of neovascular-dependent disease, weight, age, sex, and method of administration. Also, the dosage of the agent varies depending on the target tumor, tissue, graft, or organ. Generally, the agents can be orally or intravenously administered in an amount of about 0.01-1000 mg/day, based on an average weight of about 60 kg. Thus, an administration regimen could include long-term, daily treatment. By “long-term” is meant at least two weeks and, preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed., latest edition), Mack Publishing Co., Easton, Pa. The dosage can also be adjusted by the individual physician in the event of any complication.

The agents can be administered conventionally as compositions containing the active agent as a predetermined quantity of active material calculated to produce the desired therapeutic effect in association with the required diluent, i.e., carrier or vehicle. Depending on the intended mode of administration, the agent can be in pharmaceutical compositions in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. The compositions will include, as noted above, an effective amount of the selected agent in combination with a pharmaceutically acceptable carrier and, in addition, may include other medicinal agents, pharmaceutical agents, carriers, adjuvants, diluents, etc. By “pharmaceutically acceptable” is meant a material that is not biologically or otherwise undesirable, i.e., the material may be administered to an individual along with the selected agent without causing any undesirable biological effects or interacting in a deleterious manner with any of the other components of the pharmaceutical composition in which it is contained.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc. an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Thus, the compositions are administered in a manner compatible with the dosage formulation and in a therapeutically effective amount. As discussed above, precise amounts of active ingredient required to be administered depend on the judgment of the practitioner and are peculiar to each individual.

For oral administration, fine powders or granules may contain diluting, dispersing, and/or surface active agents, and may be presented in water or in a syrup, in capsules or sachets in the dry state, or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated.

Parenteral administration, if used, is generally characterized by injection. Injectables can be prepared in conventional forms, either as liquid solutions or suspensions, solid forms suitable for solution or suspension in liquid prior to injection, or as emulsions.

As used throughout, by “subject” is meant an individual. Preferably, the subject is a mammal such as a primate, and, more preferably, a human. Thus, the “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

Also provided by the present invention is a method of screening for an agent that stabilizes vasculature or promotes remodeling of vasculature, comprising the steps of culturing allantoic cells, under conditions that allow the formation and remodeling of vasculature; contacting the vasculature with the agent to be screened; detecting the remodeling of the vasculature; and comparing the remodeling in the culture to be screened with the remodeling in a control culture, less remodeling in the culture to be screened indicating an agent that stabilizes vasculature and more remodeling in the culture to be screened indicating an agent that promotes remodeling of vasculature. Vasculogenesis results in the formation of vascular networks in culture. Over time, however, the vascular networks are remodeled (i.e., become progressively less complex and revert to more primitive vascular patterns). For example, during the process of culturing allantoides from 8-8.5 day (postcoitus) mouse embryos, the level of vessel complexity decreases beyond a twenty-four hour period. The ability of an agent to stabilize the vascular networks or to promote remodeling can be screened using a culture of allantoic cells.

The present invention also further provides a method of screening for genes involved in promoting or inhibiting neovascularization, comprising the steps of culturing allantoic cells in the presence or absence of an agent that promotes or inhibits differentiation of mesodermal stem cells into endothelial cells or endothelial precursor cells or promotes or inhibits the differentiation of endothelial cell precursors into endothelial cells; isolating nucleic acids from the allantoic cells; and detecting differences in a genetic profile in the presence and absence of the agent, wherein a specific change or changes in the genetic profile indicates a gene or genes involved in promoting or inhibiting neovascularization. To produce a genetic profile, the nucleic acids are detected that are present at higher or lower levels from the allantoic cells cultured in the presence of the agent as compared to the allantoic cells cultured in the absence of the agent, wherein the nucleic acid present at higher or lower levels in allantoic cells cultured in the presence the agent indicates genes involved in promoting or inhibiting neovascularization.

The present invention also provides a method of screening for genes involved in promoting or inhibiting neovascularization, comprising the steps of culturing allantoic cells of selected developmental stages (including, for example, approximately 5.5, 6.0, 6.5, 7.0, 7.5, 8.0, 8.5, 9.0, 9.5, and 10 dpc) of neovascularization in the presence or absence of an agent that promotes or inhibits differentiation of mesodermal stem cells into endothelial cells or endothelial precursor cells or promotes or inhibits the differentiation of endothelial cell precursors into endothelial cells; isolating nucleic acids from the allantoic cells; and detecting the nucleic acids present at higher or lower levels in the allantoic cells cultured in the presence of the agent as compared to the allantoic cells cultured in the absence of the agent, or present at higher or lower levels in allantoic cells at later developmental stages compared to earlier developmental stages of neovascularization, wherein the nucleic acids present at higher or lower levels in allantoic cells cultured in the presence of the agent or in the later developmental stages indicate genes involved in promoting or inhibiting neovascularization. Stated differently, differences in a genetic profile ay various developmental stages in the presence or absence of the agent is performed, wherein a specific change or changes in the genetic profile indicates a gene or genes involved in promoting or inhibiting neovascularization. Thus, pre-neovascularization and post-neovascularization genetic profiles can be compared by following the time course of normal vascularization. Also, pre-treatment and post-treatment genetic profiles can be compared at selected developmental stages. For example, the effect of an agent that promotes either vasculogenesis or angiogensis during a period of normal vasculogenesis versus a period of normal angiogenesis can be evavluated.

The detecting step can comprise a RT-PCR or oligonucleotide microarray. The nucleic acid detected can be RNA or DNA. Methods of isolating and detecting nucleic acids are well known in the art. See e.g., Molecular Cloning, eds. Sambrook, Fritsch, and Maniatis, (1989). Optionally, following isolation of the RNA, the RNA can be reverse transcribed to cDNA using techniques well known in the art, and cDNA, rather than RNA, can be detected. Also provided is the screening method, further comprising amplifying the cDNA to produce amplification products, and comparing the amplification products of the cells cultured in the presence and absence of the agent, wherein the amplification products correlate with gene expression. The comparison of cDNA or amplification products can be performed by detecting different bands of sequence or by applying the cDNA or amplification products to gene arrays, which can be purchased commercially, for example, from Affymetrix (Santa Clara, Calif.). Additional methods of isolating RNA, reverse transcribing RNA, detecting RNA, cDNA, amplifying cDNA, and comparing cDNA and amplification products are techniques well known in the art. See, for example, Basic Cloning Procedures (Springer Lab Manual), ed. Berzins (1998) and Molecular Cloning, eds. Sambrook, Fritsch, and Maniatis, (1989), which are incorporated by reference herein.

The invention further provides a method of preventing a neovascular-dependent disease in a subject or treating a subject with a neovascular-dependent disease, comprising administering to the subject a therapeutically effective amount of either a nucleic acid that blocks expression of the gene identified by the screening method and further identified to promote neovascularization or a nucleic acid that encodes a protein that promotes expression of the gene identified by the screening method and further identified as inhibiting neovascularization. For the nucleic acid that encodes a protein that promotes expression of the gene identified by the screening method and further identified as inhibiting neovascularization, the nucleic acid must be expressed in a cell for neovascularization to be inhibited.

As used throughout, by “blocks expression” is meant any partial or complete interruption of expression of a gene, including, for example, by binding an antisense oligonucleotide or ribozyme to the gene or to an RNA transcript of the gene that increases or decreases neovascularization so as to prevent or reduce expression of the gene.

Also, provided is a method of promoting vascularization of a tissue, organ, or graft in a subject, comprising administering to the subject either a nucleic acid that blocks expression of the gene identified by the screening method and further identified to inhibit neovascularization or a nucleic acid that encodes a protein that promotes expression of the gene identified by the screening method and further identified as promoting neovascularization. In the case of the nucleic acid that encodes a protein that promotes expression of the gene identified by the screening method and further identified as promoting neovascularization, the nucleic acid is expressed in a cell and neovascularization is promoted.

In the methods that involve administering to a subject a nucleic acid, the nucleic acid can be administered to the subject in a gene delivery vehicle. The gene delivery vehicle can be a virus, which can be selected from the group consisting of adenovirus, retrovirus and adeno-associated virus. Alternatively the nucleic acid can be administered to the subject in a liposome.

It is understood that nucleic acids administered to a subject would be provided in a therapeutically effective amount by a nucleic acid gene delivery vehicle. Thus, the delivery vehicle would be administered to produce a therapeutically effective amount of the desired gene product in a particular subject.

The invention further provides a method of determining whether stem cells of unknown endothelial cell potential can be promoted to differentiate into endothelial cell precursors, comprising culturing the stem cells under conditions that allow the cells to differentiate into endothelial cell precursors; and determining the presence of endothelial cell precursors by detecting the co-expression of TAL1 and FLK1.

The present invention further provides methods of screening for an agent that modulate vasculogenesis from circulating endothelial cell precursors. By “modulate vasculogenesis” is meant promoting or inhibiting vascular growth or complexity.

In one embodiment the method comprises the steps of (a) contacting one or more embryonic vascular networks with the agent to be screened, under conditions in which extraembryonic mesodermal stem cells, or derivatives thereof, can migrate to the embryonic vascular network or networks; (b) detecting, in the vascular network or networks, endothelial cells or endothelial cell precursors derived from extraembryonic mesodermal stem cells, or derivatives thereof; and (c) comparing the endothelial cells or endothelial cell precursors derived from extraembryonic mesodermal stem cells, or derivatives thereof, in the networks contacted with the agent to be screened, with the endothelial cells or endothelial cell precursors derived from extraembryonic mesodermal stem cells, or derivatives thereof, in untreated networks.

In another embodiment, the invention provides an in vitro method of screening for an agent that promotes or inhibits vasculogenesis, comprising the steps of (a) co-culturing extraembryonic mesodermal stem cells and intraembryonic mesodermal stem cells, under conditions that allow formation of one or more vascular networks; (b) contacting the co-culture with the agent to be screened; (c) detecting, in one or more vascular networks, endothelial cells or endothelial cell precursors derived from extraembryonic mesodermal stem cells, or derivatives thereof; and (d) comparing the endothelial cells or endothelial cell precursors derived from extraembryonic mesodermal stem cells, or derivatives thereof, in the vascular network or networks in the culture contacted with the agent to be screened, with the endothelial cells or endothelial cell precursors derived from extraembryonic mesodermal stem cells, or derivatives thereof, in the vascular network or networks of the untreated cultures.

In the embodiments of the method of screening for agents that modulate vasculogenesis from circulating endothelial cell precursors, an increase in the number of endothelial cells or endothelial cell precursors derived from extraembryonic mesodermal stem cells, or derivatives thereof, in the vascular networks in the presence of the agent to be screened indicates an agent that promotes vasculogenesis; whereas, a decrease in endothelial cells or endothelial cell precursors derived from extraembryonic mesodermal stem cells, or derivatives thereof, in the vascular networks in the presence of the agent to be screened indicates an agent that inhibits vasculogenesis.

Agents that increase endothelial cells or endothelial cell precursors derived from circulating endothelial cell precursors, including for example, extraembryonic mesodermal stem cells or derivatives thereof, can act by several different mechanisms or combination of mechanisms. For example, promoting agents can attract or otherwise promote the migration of circulating endothelial cell precursors thereof to the site of blood vessel formation. Such agents include agents that promote inflammatory processes (e.g., tumor necrosis factor alpha,IL-1, IL-6, IL-12, IL-5, IL-4, IL-13, IL-1beta, carrageenan, IFN-gamma). (See, e.g., Raz, A. et al. 2000; Samaniego, F. et al. 1997.) As another example, agents that promote vasculogenesis can promote cell division of endothelial cell precursors. Markers that are upregulated by promoting agents and downregulated by inhibiting agents include intracellular adhesion molecule-1 (ICAM-1), P-selectin, and vascular cell adhesion molecule-1 (VCAM-1), L-selectin, E-selectin, integrin CD11b/CD18. (See, e.g., Rozdzinski, E. et al, 1995.)

Agents that decrease endothelial cells or endothelial cell precursors derived from circulating endothelial cell precursors include agents that repel or otherwise inhibit migration of the circulating endothelial cell precursors, to the site of blood vessel formation. Such agents include agents that inhibit inflammatory processes (e.g., olopatadine (Pantanol), D-hormone (alfacalcidol and clacitriol), inodmethacin (cox inhibitor, which abrogates carrageenan), bradykinin antagonists, cyclooxygenases 1 and 2, filamentous hemagglutinin of Brodatella pertussis (FHA, which inhibits integrin CD11b/CD18 binding). (See, e.g., Cook, E. B. et al., 2000; Scacht, E. Z., 2000; Raz, A. et al., 2000; Hsieh and Stewart, 1999; Cronstein BN et al., 1999; Rozdzinski, E. et al. 1995). Agents that decrease endothelial cells or endothelial cell precursors derived from circulating endothelial cell precursors include agents that reduce cell division of circulating endothelial cell precursors at the site of blood vessel formation.

“Circulating endothelial cell precursors” can include cells of intraembryonic mesodermal stem cell origin or extraembryonic mesodermal stem cell origin. “Extraembryonic mesodermal stem cells” can include, for example, cells from extraembryonic blood islands of the same embryo. Preferably, the extraembryonic mesodermal stem cells are splanchnic mesodermal stem cells, more preferably, mammalian splanchnic mesodermal stem cells. Even more preferably, the extraembryonic splanchnic mesodermal stem cells are allantoic mesodermal stem cells. “Extraembryonic mesodermal stem cells” can also include, for example, cells derived from an embryo other than the embryo serving as the source of the embryonic vascular networks. More specifically, mesodermal stem cells from the paraaortic region of the chicken embryo could be transplanted into or provided to a different chicken embryo or a non-chicken embryo, including, for example, a quail or mouse embryo. Alternatively, mesodermal stem cells from the aorta-gonad-mesonephros (AGM) region of a mouse embryo could be transplanted into or provided to a different mouse embryo or a non-murine embryo, including, for example, a quail or chick embryo, to form a chimera. In yet another embodiment, the extraembryonic mesodermal stem cells can be derived from bone marrow of the same or a different species from the species that is the source of the embryonic vascular networks.

By “derivatives” of extraembryonic mesodermal stem cells is meant one or more cells that arise from an extraembryonic mesodermal stem cell, including, for example, endothelial cell precursors of various stages.

Preferably, the extraembryonic mesodermal stem cells used in the present methods comprise a detectable tag, including for example, a specific antigen or fluorescent label. Thus, the extraembryonic mesodermal stem cells are specifically labeled so that the extraembryonic mesodermal stem cells and their lineage can be distinguished from intraembryonic stem cells and their lineage. The extraembryonic stem cells can be labeled in situ, in vivo, ex vivo or in vitro. For example, extraembryonic stem cells can be labeled in a living embryo using DiI, Celltracker green (Molecular Probes, Eugene, Oreg.), Syto green (Molecular Probes, Eugene, Oreg.), or a retrovirus (such as a human histone 2B promoter driving expression of Green Fluorescent Protein (“GFP”)/Yellow Fluorescent Protein (“YFP”) or driving expression of YFP). In another embodiment, extraembryonic stem cells can be labeled in a living embryo by transfecting (e.g., by electroporation, lipofectamine, or other techniques know in the art) the extraembryonic stem cells with a GFP or YFP expression plasmid. In yet another embodiment, the extraembryonic mesodermal stem cells can be introduced from intraembryonic or extraembryonic tissues of an embryo other than the embryo serving as the source of the embryonic vascular networks, and markers specific for the extraembryonic mesodermal stem cells can be used to label the extraembryonic stem cells and their lineage. For example, extraembryonic stem cells derived from a transgenic mouse embryo could be transplanted or otherwise provided to vascular networks of an embryo of a different species or of a control embryo of the same species. For example, the extraembryonic stem cells can be derived from TIE-2-lacZ mice having TIE-2 promoter (endothelial cell specific) linked to lacZ; ROSA-26 mice derived from ES cells bearing a retroviral insertion of β-gal gene; TIE-2-GFP mice having TIE-2 promoter linked to GFP; TIE-2-GFP-ROSA-26 mice that are chimeras in which endothelial cells express GFP and all cells express lac-Z.

In one embodiment, the embryonic vascular networks used in the present method are derived from an early stage embryo, wherein the early stage embryo has one source of extraembryonic mesodermal stem cells. More specifically, the source of extraembryonic mesodermal stem cells in one embodiment is blood islands in yolk sac, and, in another embodiment the source is the allantois, which is comprised of extraembryonic splanchnic mesodermal cell. Preferably, the early stage embryo is selected from a primitive streak stage up to six somite stage embryo. Even more preferably, the early stage embryo is selected from a primitive streak embryo, a one somite embryo, a two somite embryo, a three somite embryo, or any stage in between.

In the various embodiments of this invention, the endothelial cells or endothelial cell precursors are preferably detected by one or more endothelial cell markers. The marker is preferably selected from the group consisting of TAL1, Flk1, CD34, VE-cadherin, Tie 2, platelet/endothelial cell adhesion molecule (PECAM), QH1, or other endothelial cell markers known in the art.

In one embodiment, the contacting step is performed in the whole embryo, in vivo. In an alternative embodiment, the contacting step is performed in vitro.

The present invention also provides a method of promoting vasculogenesis in a tissue or organ, comprising contacting the tissue or organ with a therapeutically effective amount of the agent identified by the methods of screening for an agent that promotes vasculogenesis from circulating endothelial cell precursors. Further provided is a method of inhibiting vasculogenesis using an agent identified by the method of the invention as an agent the inhibits vasculogenesis from circulating endothelial cell precursors is identified. The present invention also provides a method of treating a vasculogenic-dependent disease in a subject, comprising administering to the subject an agent identified by the screening method as an agent that inhibits vasculogenesis. The treatment method comprises contacting a therapeutically effective amount of the agent with the tissue, organ, or tumor where vasculogenesis is sought to be inhibited. The contacting step can be in vivo or in vitro. The dosages and methods of administration are determined as described above.

The present invention is more particularly described in the following examples which are intended as illustrative only since numerous modifications and variations therein will be apparent to those skilled in the art.

### EXAMPLES

### Example 1

### Characterization of Vascular Marker Proteins in Allantoic Neovascularization

A novel protocol was used that rendered the normally curved or lordotic mouse embryo into a planar format. This procedure, combined with capability of the confocal microscope that is able to represent all embryonic vessels in a single image, facilitated analysis of vascular patterns and developmental gradients. The data provide a number of new insights into the processes of vasculogenesis and hematopoiesis that include a more detailed understanding of the relationship between TAL1 and FLK1 expression in these lineages.

### Antibodies

Rabbit polyclonal anti-mouse TAL1/SCL (Kallianpur et al. 1994) was obtained from Stephen J. Brandt (Vanderbilt University and Veterans Affairs Medical Center, Nashville, Tenn.). Rabbit anti-mouse Flk1 (Shalaby et al, 1995) was provided by Andre Schuh (University of Toronto, Toronto, Ontario, Canada). Rabbit anti-mouse CD34 (Baumhueter et al (1993) was provided by Lawrence Lasky (Genentech, Inc., San Francisco, Calif.). Rat monoclonal anti-mouse Tie2 (Koblizek et al (1997) was obtained from Steven Stacker (Ludwig Institute for Cancer Research, Victoria, Australia). Rat monoclonal antibodies to recombinant VE-cadherin (clone 19E6) (Corada et al. 1999) were provided by Elisabetta Dejana and Maria Lampugnani (Istituto di Ricerche Farmacologiche Mario Negri, Milano Italy). Rat anti-mouse PECAM monoclonal antibodies were purchased from PharMingen (San Diego, Calif.).

### Wholemount Immunolabeling

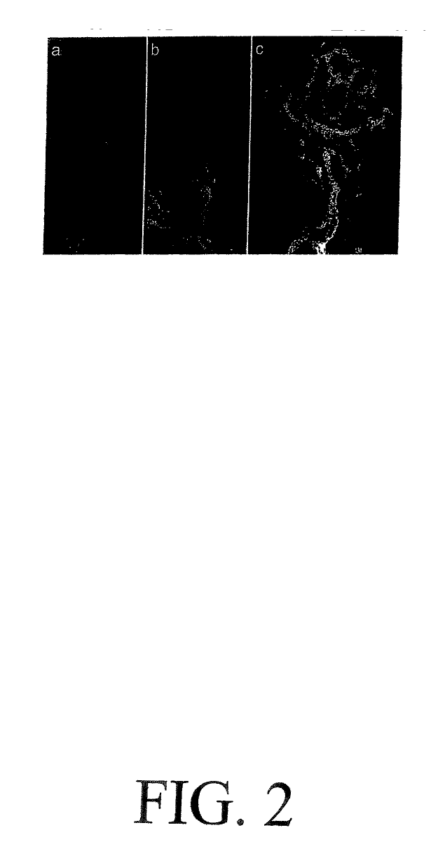

For imunolabeling for TAL1, Flk1, CD31, and CD34, embryos at 7.0-9.5 dpc (0.5 dpc, plug date) were dissected free of the uterine muscle and decidua and placed into EPBS (4° C.). Reichert's membrane and the ectoplacental cone were removed and the embryos flattened by cutting the yolk sac lateral to the embryonic axis and removing the amniotic sac (FIG. 2). Fixation was by infusion of 3% paraformaldehyde into the EPBS (5 minutes) followed by fixation in 3% paraformaldehyde (10 minutes). Embryos were permeabilized in PBSA containing 0.02% Triton-X 100 (30 minutes), exposed to a blocking solution, 3% BSA/PBSA, and then to appropriate primary and secondary antibodies (Jackson Immuno Research Laboratories, Inc., West Grove, Pa.). Incubations were for a period 12-18 hours at 4° C. Embryos were mounted ventral side up using an antiphotobleaching medium. See Giloh (1982). Immunolabeling for VE-cadherin and Tie2 was as described above except that embryos were exposed to primary antibodies prior to fixation (1.5 hours, 4° C.).

### Allantois Culture and Immunolabeling

Allantoides of 7.5dpc embryos were excised, washed in EPBS (4° C.) and then pipetted into Nunc 4 chambered culture slides (Fisher Scientific Co., Suwanee, Ga.) containing 0.4 ml of DMEM, 10% Fetal Bovine Serum, and 1% Penicillin Streptomycin. Explants were cultured at 37° C. in a 5% CO₂ incubator for 12-20 hours and then fixed and permeabilized as described above. The explants were blocked in 3% BSA/PBSA 12-18 hrs, exposed to PECAM antibodies (1.5 hours, 26° C.), washed 3×40 minutes in PBSA, incubated in appropriate secondary antibodies (1.5 hours, 26° C.), washed in PBSA 3×30 minutes, and mounted as described above.

### Microscopy and Image Processing

Embryos were analyzed using a Bio-Rad MRC 1024 Laser Scanning Confocal Microscope (Bio-Rad Microscopy Division, Cambridge, Mass.). Optical sectioning along the dorsoventral axis (Z-axis) was performed and the images collapsed into a single focal plane using manufacturer's software. Differential Interference Contrast (DIC) images were generated using a research grade Leitz™ photomicroscope equipped with a Photometrics™ (Tucson, Ariz.) Quantix CCD camera. Images were processed using NIH Image 1.62 software (NIH, Bethesda, Md.) and Adobe Photoshop 5.0 (Adobe Systems, Inc., San Jose, Calif.).

### Characterization of the Angioblast and the Hematopoietic Cell Phenotype

Initial characterization of the angioblast was conducted in 8.3 days postcoitum (dpc) embryos, a stage when both established and forming vessels are present. Double immunofluorescence demonstrated that TAL1 and Flk1 co-labeled endothelial cells of morphologically identifiable vessels as well as dispersed populations of mesodermal cells. To pursue the possibility that the dispersed TAL1⁺/Flk1⁺ cells represent the progenitors of endothelial cells, blood vessel development was followed in 6.5-7.0 dpc embryos. At 6.5 dpc, dispersed TAL1⁺/Flk1⁺ mesodermal cells were detected in extraembryonic regions. When the corresponding regions of 7.0-7.3 dpc embryos were examined, polygonal arrangements of small caliber vessels (primary vascular networks) were evident in the regions previously populated by the TAL1⁺/Flk1⁺ cells. These data show that TAL1⁺/Flk1⁺ cells (angioblasts) are the precursors of endothelial cells.^{8,9}

To characterize extraembryonic hematopoietic cells, TAL1 and Flk1 immunofluorescence was followed in 6.5-7.0 dpc embryos. At 6.5 dpc blood islands were characterized by intense TAL1 and weak Flk1 immunostaining. A similar pattern of expression was evident in the blood islands at 7.0-7.3 dpc. Analysis of optical sections demonstrated that endothelial cells which comprise the outer component of the blood island were Flk1⁺ while cells representing the hematopoietic lineage, those forming the “core,” were Flk1⁻. Based on these data it is concluded that extraembryonic hematopoietic cells are TAL1⁺/Flk1⁻.

### Intraembryonic Vasculogenesis 6.5-8.0 dpc:TAL1, Flk1 and PECAM Expression

Intraembryonic vasculogenesis is initiated in the cranial region of 7.3 dpc embryos. Evident cranially were two populations of Flk1⁺ and TAL1⁺ cells that were joined across the midline by a “string” of cells forming a crescent. The bi-lateral distribution of the TAL1⁺/Flk1⁺ cells coincides with regions of the embryo that are fated to give rise to the heart (Tam and Behringer 1997) suggesting that the TAL1⁺/Flk1⁺ cells are endocardial progenitors.

The interval between 7.0 and 7.8 dpc is an active period of vasculogenesis. During this period, TAL1⁺ and Flk1⁺ cell numbers increase dramatically and the aortic primordia first become discernible. The first intraembryonic PECAM immunofluorescence was localized to the aortic primordia of 7.8 dpc embryos. Comparison of PECAM immunostaining to that of TAL1 and Flk1 demonstrates that PECAM is not expressed by all TAL1⁺/Flk1⁺ cells. These data establish that TAL1 and Flk1 are expressed earlier than PECAM and suggests that angioblasts, isolated TAL1⁺/Flk1⁺ cells, do not express PECAM.

### Allantoic Vasculogenesis: TAL1, Flk1 and PECAM Expression

Initial blood vessel formation in the allantois is indicated by the presence of a small number of dispersed TAL1⁺/Flk1⁺ cells at 7.0 dpc. By 7.3-7.5 dpc, TAL1⁺/Flk1⁺ cells are numerous. At this stage, no organized blood vessels or vessel primordia could be detected. By 8.3 dpc PECAM immunofluorescence indicated the presence of both vessel primordia and vascular networks in the allantois.

To investigate whether these vessels arise by vasculogenesis, or by angiogenesis, allantoides were isolated and cultured. After 12 hours in culture, PECAM staining revealed both vessel primordia and vascular networks. Since these vessels arose from tissue containing TAL1⁺/Flk1⁺ cells but no organized blood vessels, it can be concluded that neovascularization occurred via vasculogenesis and that the TAL1⁺/Flk1⁺ cells are the precursors of endothelial cells.

### TAL1, Flk1, PECAM, CD34, VE-Cadherin and Tie2 Expression in Intraembryonic Vasculogenesis: 8.0-8.5 dpc

Between 8.0 and 8.5 dpc, a rudimentary circulatory system is established. The expression patterns of TAL1, Flk1, PECAM, CD34, VE-cadherin and Tie2 in the vessels of 8.2-8.3 dpc and 8.5 embryos in prominent morphological structures of the circulatory system such as the bilateral aortae, the endocardial primordia and primary vascular networks that form lateral to the embryonic axis, which are referred to as lateral vascular networks, are summarized in Table 1.

TABLE 1
Expression of TAL1, Flk1, PECAM, CD34, VE-cadherin, and Tie2
during intraembryonic vasculogenesis

| | 8.2-8.3 dpc | | | 8.5 dpc | | |
|---|---|---|---|---|---|---|
| | (4-6 somites) Embryos | | | (7-10 somites) Embryos | | |
| | | | Lateral | | | Lateral |
| | Endo- | Dorsal | Vascular | Endo- | Doral | Vascular |
| Protein: | cardium | Aortae | Networks | cardium | Aortae | Networks |
| TAL1 | + | + | + | − | + | + |
| Flk1 | + | + | + | + | + | + |
| PECAM | + | + | − | + | + | − |
| CD34 | − | + | − | + | + | − |
| VE- | − | + | − | + | + | − |
| cadherin | | | | | | |
| Tie2 | − | − | − | + | + | − |

As described above, endocardiogenesis is initiated at 7.3 dpc. Between 8.2 and 8.5 dpc the bilateral heart fields are translocated to the midline forming the definitive endocardium. At 8.2-8.3 dpc, Flk1 expression was observed throughout the merging heart fields. In contrast, while TAL1 expression was associated with the caudal portions of the heart fields, those lying along the anterior intestinal portal, only weak staining was detected in the more cranial portions of the fields. At 8.5 dpc, the endocardium is characterized by strong Flk1 immunofluorescence and the absence of detectable TAL1 immunofluorescence. Unlike TAL1, immunofluorescence associated with PECAM, CD34, VE-cadherin and Tie2 was readily detected on the endocardium. It is concluded from these data that the TAL1⁺/Flk1⁺ cells observed in cranial regions at 7.3 dpc and in heart fields at 8.2 dpc, represent the progenitors of the TAL1⁻/Flk1⁺ endocardial endothelial cells seen at 8.5 dpc.

### TAL1, Flk1, PECAM, CD34, VE-Cadherin and Tie2 Expression in the Dorsal Aortae: 8.2-8.5 dpc

The dorsal aorta is derived form the fusion of bilateral primordia, the dorsal aortae. At 8.3 dpc both cranial and caudal portions of the dorsal aortae exhibited intense PECAM staining, while the more intermediate portion stained less intensely. This immunostaining pattern coincided with morphogenetic features of the developing aortae. Intense PECAM staining was associated with segments that, based on physical sections, had a defined lumen while less intense staining was detected in segments composed of primary vascular networks. It is concluded that the aortae form in a bi-directional manner and that vascular networks are an essential component of aortic morphogenesis. Similar to PECAM, immunostaining for TAL1, Flk1, CD34 and VE-cadherin was localized to the aortic primordia of 8.2 and 8.5 dpc embryos. In contrast to these proteins, Tie2 immunofluorescence was absent at 8.2 dpc; however, expression was detected at 8.5 dpc. This observation suggests that Tie2 expression correlates with a discrete step in vessel maturation.

### TAL1, Flk1, PECAM, CD34, VE-Cadherin and Tie2 Expression in the Lateral Vascular Networks: 8.2-8.5 dpc

Between 8.2 and 8.5 dpc the lateral vascular networks are formed. These networks extend from a region just lateral to the aortae to an ill-defined boundary where they connect with the extraembryonic vasculature. Isolated TAL1⁺/Flk1⁺ cells can be detected within the lateral regions as early as 7.6 dpc, by 8.2 dpc the first networks are apparent and by 8.5 dpc the lateral vascular networks are clearly discernible. Double immunofluorescence experiments revealed that TAL1 and Flk1 are co-expressed in cells of both the forming and established lateral vascular networks. In contrast to the expression of TAL1 and Flk1, PECAM expression was conspicuously absent in these vessels at both 8.2 dpc and 8.5 dpc. The immunostaining patterns of CD34 and VE-cadherin at 8.2 and 8.5 dpc were similar to that of PECAM, with expression associated with the forming aortae but absent in the lateral vascular networks.

The absence of PECAM, CD34 and VE-cadherin expression in the lateral vascular networks at 8.5 dpc was unexpected, as each of these proteins were associated with the morphogenesis/maturation of other primary vascular networks (i.e., in the developing allantois and aortae). This finding was pursued in double immunofluorescence studies. Immunolabeling of 8.5 dpc embryos with TAL1 and PECAM antibodies demonstrated co-labeling of the aortic primordium and the absence of PECAM expression in the TAL1⁻ cells of lateral vascular networks. Double-immunolabeling studies using Flk1 and PECAM antibodies yielded similar results These data established that cells of the aortic primordia are TAL1⁺/Flk1⁺/PECAM⁻ while those of the lateral vascular networks are TAL1⁺/Flk1⁺/PECAM⁻. Similar studies comparing TAL1 and Flk1 expression to that of either CD34 or VE-cadherin demonstrated that co-expression of TAL1 and Flk1 was confined to the aortae while laterally, only TAL1⁺/Flk1⁺ cells were detected.

To determine if the absence of PECAM, CD34 and VE-cadherin expression had morphological consequences, vasculogenesis in the lateral regions was evaluated using Flk1 antibodies. Analysis of Flk1 immunostaining indicated that vascular morphogenesis, including those events requiring endothelial cell-cell adhesion, had proceeded normally. As part of this analysis, a population of Flk1⁺ and TAL1⁺ cells located along the lateral margin of the aortae were detected. The position of these TAL1⁺/Flk1⁺ cells is consistent with the possibility that such cells are angioblasts, some of which seem to be in the process of “joining” the developing aortae.

While PECAM, CD34 and VE-cadherin were each expressed by cells of the aortic primordia, differences in their temporal and spatial immunofluorescence patterns were observed. For instance, PECAM expression on the aortic primordia was initially associated with the entire cell surface while later expression was localized to sites of cell-cell contact. In contrast, VE-cadherin expression, when observed, was always present at sites of cell-cell contact.

### TAL1 is Down-Regulated as Part of Endothelial Cell Maturation

The diminution of TAL1 expression associated with endocardial development suggested a relationship between the level of TAL1 expression and the state of endothelial cell maturation. To investigate this possibility, TAL1 expression was followed during aortic development. While strong TAL1 immunofluorescence was associated with the aortae of 8.2 and 8.4 dpc embryos, by 9.0 dpc no expression was detected. Expression of TAL1, Flk1 and PECAM in the aortae of 9.0 dpc embryos was examined in triple immunofluorescence studies. In contrast to the uniform expression of PECAM on endothelial cells, TAL1 immunofluorescence on a segment of an aortae and the associated intersomitic and intervertebral vessels was confined to a population of uniformly round cells. Analysis of optical sections demonstrated that these cells were confined to the vascular lumen suggesting that they are associated with the hematopoietic rather than the endothelial lineage. When the TAL1 and PECAM immunostaining patterns are superimposed, the lack of detectable TAL1 expression in endothelial cells was evident. Flk1 expression was examined to determine if a correlation exists between the level of TAL1 expression and that of Flk1. Clear Flk1 immunofluorescence was associated with endothelial cells. Comparison of TAL1 and Flk1 expression establishes that mature endothelial cells are TAL1⁻/Flk1⁺. The ability to detect FLK1 protein in endothelial cells lacking TAL1 expression suggests that the expressions of these proteins are independently regulated.

### Example 2

### Effect of FLT-1 on De Novo Vascular Development in the Allantois

As described in Example 1, 7.0-8.0 dpc embryos were dissected from pregnant female mice into cold (4° C.) sterile Dulbecco's PBS, and the allantoides were dissected away from each embryo and placed in cold (4° C.) sterile Dulbecco's PBS. The allantoides were transferred to fibronectin-coated (50 μg/ml) culture dishes (Nunc) containing DMEM, 10% FBS, 1% pen-strep/glutamine alone or with soluble FLT-1 or other agent to be screened. Soluble FLT-1 (chimeric proteins composed of FLT-1 ectodomain fused to Ig Fc region) was added to the allantois cultures at a concentration of (4 μg/ml) and incubated for 24 h.

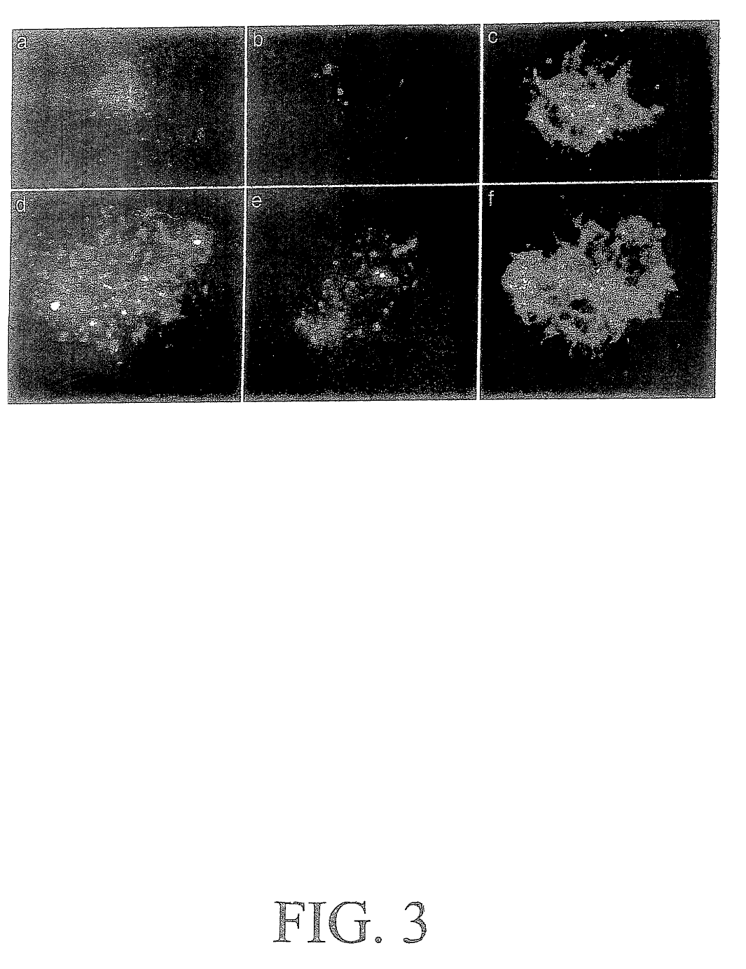

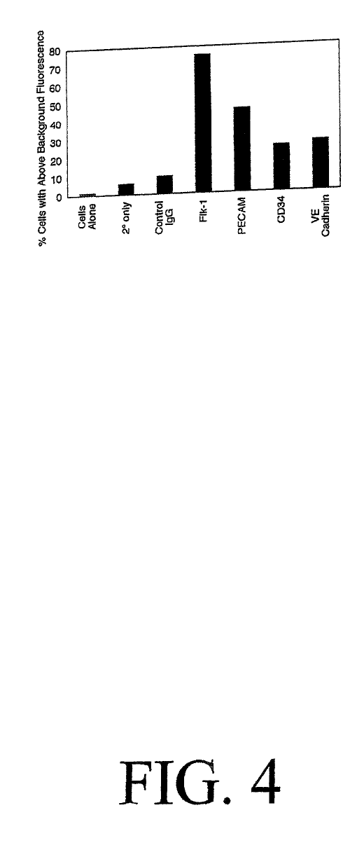

The allantoides were cultured for varying periods of time (12, 24 and 36 h) at 37° C., 5% CO₂ and subsequently fixed and processed for imunohistochemistry and confocal analysis as described above. The allantoides were immunolabeled with anti-TAL1, anti-FLK-1 and anti-PECAM/CD34). The results showed a disruption in vascular development as compared to allantoides cultured in medium alone. See FIG. 3.

### Example 3

### Effect of VEGF on De Novo Vascular Development in the Allantois

Allantoides were isolated, cultured, and analyzed according to the general methods described in Example 2, except that, instead of FLT-1, VEGF was added to the culture medium. Incubation of allantois cultures with recombinant VEGF (2 μg/ml) for 24 hr resulted in a hyperfused vascular phenotype similar to that described in in vivo studies by Drake et al. 1995. See FIG. 3. It should be noted that the hyperfusion-promoting effects of exogenously added VEGF can be observed earlier than 24 h post treatment.

### Example 4

### Flow Cytometric Analysis

The cells of 8.0-8.5 dpc mouse allantoides were dissociated into a single cell suspension using trypsin, EDTA. The cells were then washed and the protease neutralized by addition of soybean trypsin inhibitor or 10% serum. The cells were centrifuge at 700× g for 5 minutes. Optionally, the cell suspension can be passed through a screen. The cells were washed and allowed to recover in complete medium for 30 min at 37° C., 5% CO₂. The cells were then incubated with medium containing serum of the same species of the secondary antibody (e.g., donkey serum). Optionally, the cells can be counted using hemacytometer (optional). The cell suspension was subsequently aliquoted into as many tubes as antibodies or control to be used. For example, seven tubes were prepared for control samples in the absence of primary antibody (cells alone, secondary antibody only, and control IgG) and for experimental samples with primary antibodies (anti-FLK1, anti-PECAM, anti-CD34, anti-VE-cadherin). The control and experimental samples were placed on ice and incubated with primary antibodies at 4° C. for 0.5-1 hr. The samples were centrifuged, washed with PBS (4° C.), and incubated with fluorochrome-labeled secondary antibody for 0.5-1 hr. Following incubation with the secondary antibody, the samples were centrifuged, washed, and subject to flow cytometry analysis using techniques known in the art.

### Example 5

### Analysis of Vascular Stabilization and Remodeling

To analyze the capacity of an agent to stabilize the preexisting vasculature or to accelerate the remodeling process, allantoides explants from 8-8.5 dpc mice were prepared and cultured as described above. Some of the explants, however, were cultured in the presence or absence of anti-CD34 (20 μg/ml) for 24 hr. The explants were subsequently fixed and processed for immunohistochemistry using anti-PECAM to visualize the vascular pattern as described above. In the absence of an exogenous agent like anti-CD34, the vasculature of the allantois in culture over the 24 hour culture period undergoes a remodeling in which the central vessel with an elaborate vascular network remodels to form a simple uniform vascular network (i.e., a morphologically more primitive pattern, composed of many small caliber vessels with lumens). In the presence of anti-CD34 this remodeling is perturbed. Instead of observing the uniform vascular network that occurs with culturing, the vascular pattern is disrupted in the presence of anti-CD34 to show a reduction in uniformity (i.e., disconnected vascular networks). This perturbation is interpreted as a destabilizing effect.

### Example 6

### Assay of Vasculogenesis in Allantoides Cell Spheroids

Vasculogenic spheriods/mesodermal aggregates derived from dissociated allantoic mesodermal cells are also used to screen for compounds/drugs that modulate blood vessel formation. Allantoides from 7.5 dpc embryos from a pregnant female mice are dissected as described above and are placed in cold (4° C.) sterile Dulbecco's PBS. The allantoides are then transferred to trypsin-EDTA dissociation medium and incubate for approximately 10 minutes and, optionally, passed through a 35 μm screen. The trypsin is neutralized by washing cells either with serum containing DMEM or DMEM containing soybean trypsin inhibitor (0.5 mg/ml). The cells are then resuspended in DMEM and then in DMEM containing 1% methocel. The cell suspension is, optionally, passed through a 35 μm screen. The cells are counted using a hemocytometer. A 0.5 ml sample of the cell suspension (containing 1×10⁶ cells/ml) is placed into wells of 24 well, round-bottom (non-tissue culture coated). The cells are cultured for at least 20 hr at 37° C., 5% CO₂ with rotational shaking at 200 rpm to allow the formation of cell aggregates.

### Example 7

### Characterization of Neovascularization in Adult Mice

Transgenic mice in which Green Fluorescent Protein (GFP) is expressed under the endothelial specific promoter Tie2 have “green” endothelium. These mice undergo X-ray radiation (one exposure to a single 9.0 Gy dose of total body radiation) to eliminate their bone marrow. After X-ray radiation, the bone marrow from normal mice is transplanted into radiated Tie2/GFP mice. Bone marrow, which is obtained by aspiration from either the femur or tibia of the normal mice, is suspended in culture media, and a highly concentrated bone marrow cell suspension is injected into the recipient mouse tail vein. The resulting chimeric mice have “green” endothelial cells and “white” bone marrow.

Alternatively, a Rosa26 chimera is generated. Rosa26 mice express Lac Z in all of their cells. The Lac Z can be detected in an assay that turns Lac Z expressing cells blue. Normal mice with “white” endothelium undergo X-ray radiation to eliminate their bone marrow. After X-ray radiation, the “blue” bone marrow from transgenic Rosa26 mice is injected into the tail veins of radiated normal mice. The resulting chimeric mice will have “white” endothelial cells and “blue” bone marrow.

In “green” chimeric mice, the presence of blood vessels consisting of only “green” endothelial cells indicates the occurrence of angiogenesis alone, whereas a mixed population indicates that both angiogenesis and vasculogenesis occurred and the absence of “green” stained cells indicates adult neovascularization via vasculogenesis only. In “blue” chimeric mice, the presence of blood vessels consisting of only Lac Z positive endothelial cells indicates adult vasculogenesis. A mixed population indicates both angiogenesis and vasculogenesis, and the absence of blue stained cells indicates the occurrence of angiogenesis alone.

### Example 8

### Identification of Endothelial Cell Precursors (Angioblasts) in Bone Marrow and Blood Following Induction of Neovascularization

Three different assays are used for studying adult neovascularization. For the corneal pocket assay, the chimeric or control mice are anesthetized and a small cut is made in the cornea. Using a spatula, a small pocket is formed and a Metylcellulose pellet containing VEGF is placed in the pocket. Neovascularization is estimated visually under a microscope daily, and after 3 and 7 days mice are sacrificed for morphological analysis. For the matrigel assay, matrigel supplemented with VEGF is injected into mice subcutaneously. After 1 week the mouse is sacrificed, and the matrigel and surrounding tissues are removed for morphological analysis. For the GelFoam sponge assay, the GelFoam, which is composed of collagen type I, is soaked in VEGF and implanted subcutaneously into anesthetized mice by making a small incision in the skin. After 7 days, the sponge and surrounding tissue is removed for morphological analysis.

Bone marrow of “normal” chimeric control and chimeric neovascular induced mice, (mice employed in a neovascularization assays), are examined for the presence of TAL1/Flk1 positive cells, the presence of TAL/Flk positive cells indicating that adult bone marrow contains angioblasts.

Peripheral blood from normal, chimeric control, and chimeric neovascular induced mice is examined for the presence of TAL1/FIk1 positive cells. Briefly, blood is collected from the femoral artery and smeared on glass slides, dried, fixed and immunostained with antibodies to TAL1 and Flk1. The presence of TAL+/Flk+cells demonstrates that angioblasts are present in the circulation of neovascular induced mice. Negative results can indicate that mobilized circulated cells are still mesodermal stem cells, which, only after recruitment into an area of neovascularization, differentiate into angioblasts.

### Example 9

### Identification of Endothelial Cell Precursors (Angioblasts) in Bone Marrow and Blood Following Induction of Neovascularization

Human breast carcinoma cell lines (MDA23 1, MDA468 or SKBr3) are used to produce tumors. Initially, the cells are propagated in plastic cell culture dishes and, ut-ilizing a shaking procedure, spheroids are generated for microinjection. Either human breast cancer tissue or cell spheroids, generated from cultured breast cancer cell lines and diluted in 0.25 ml culture medium, are injected subcutaneously into nude mice. Cancerous nude mice or transgenic mice that spontaneously develop breast carcinoma undergo X-ray radiation to eliminate their bone marrow cells. “Blue” bone marrow cells from transgenic Rosa26 mice is then injected into the tail veins of the irradiated mice. In chimeric mice with spontaneous breast carcinoma, the presence of only “blue” endothelial cells in the blood vessel indicates tumor vascularization via vasculogenesis, whereas a mixed population indicates that both angiogenesis and vasculogenesis occurred and the absence of “blue” stained cells indicates tumor angiogenesis and not vasculogenesis. In nude mice, the presence of only Lac Z positive endothelial cells in blood vessels indicates vasculogenesis and an absence of tumor angiogenesis. A mixed population indicates that both angiogenesis and vasculogenesis had occurred, whereas the absence of “blue” stained cells indicates that tumor angiogenesis alone had occurred.

### Example 10

### Cells from Extraembryonic Blood Islands Contribute to Formation of Intraembryonic Blood Vessels.

Primitive streak quail embryos were isolated using techniques well-known in the art. The retrovirus GFP/YFP-H2B, a human histone 2B promoter driving expression of GFP/YFP, was microinjected into the blood islands of the embryos. The retrovirus was provided by Dr. Rusty Lansford, California Institute of Technology, Pasadena, Calif. The embryos were cultured for 12 hours using standard culture conditions. The embryos were subsequently fixed, labeled with anti-QH1 (a quail specific endothelial cell marker). Bound anti-QH1 was visualized using FITC-labeled donkey anti-mouse IgG and confocal microscopy. Specifically, intraembryonic blood vessels were identified by QH1 reactivity. The labeled blood vessels were examined to determine whether QH1 reactive cells contained GFP/YFP-H2B infected nuclei.

GFP/YFP-H2B positive cells were present in the intraembryonic vasculature of 10.5 somite embryos. For example, the left sinus venosus included GFP/YFP-H2B positive cells within the blood vessel lumena. Additionally, cells positive for both GFP/YFP-H2B and QH1 were incorporated into the blood vessel walls. These data indicate that cells from extraembryonic blood islands migrate to the site of intraembryonic blood vessel formation and contribute to intraembryonic vasculogenesis. Importantly, the precursor cells from extraembryonic blood islands require a vascular connection to migrate to a site of intraembryonic vessel formation.

Throughout this application, various publications are referenced. The disclosures of these publications in their entireties are hereby incorporated by reference into this application in order to more fully describe the state of the art to which this invention pertains.

Although the present invention has been described with reference to specific details of certain embodiments thereof, it is not intended that such details should be regarded as limitations upon the scope of the invention except as and to the extent that they are included in the accompanying claims.

### REFERENCES

1. Basic Cloning Procedures (Springer Lab Manual), ed. Berzins (1998).

2. Baumhueter S, Singer M S, Henzel W, Hemmerich S, Renz M, Rosen S D, Lasky L A: Binding of L-Selectin to the vascular sialomucin CD34. Science 262:436, 1993.

3. Cook E B, Stahl J L, Barney N P, Graziano F M. Olopatadine inhibits TNFalpha release from human conjunctival mast cells. Ann Allergy Asthma Immunol. May 2000; 84(5):504-8.

4. Corada M, Mariotti M, Thurston G, et al.: Vascular endothelial-cadherin is an important determinant of microvascular integrity in vivo. Proc Natl Acad Sci USA. Aug. 17, 1999;96(17):9815-20.

5. Cronstein B N, Montesinos M C, Weissmann G. Sites of action for future therapy: an adenosine-dependent mechanism by which aspirin retains its antiinflammatory activity in cyclooxygenase-2 and NFkappaB knockout mice. Osteoarthritis Cartilage. July 1999; 7(4):361-3.

6. Drake C J, Little C D. Exogenous vascular endothelial growth factor induces malformed and hyperfused vessels during embryonic neovascularization. Proc Natl Acad Sci USA Aug. 15, 1995;92(17):7657-61.

7. Folkman, 1990, What is the evidence that tumors are angiogenesis dependent? J Natl Cancer Inst. Jan. 3, 1990;82(1):4-6.

8. Folkman & Shing, 1992, Angiogenesis. J. Biological Chem. 267(16): 10931 -34.

9. Giloh H, Sedat J W: Fluorescence microscopy: Reduced photobleaching of rhodamine and fluorescein protein conjugates by n-propylgallate. Science 217:1252, 1982.

10. Hsieh K H, Stewart J M. Cyclic and linear bradykinin analogues: implications for B2 antagonist design. J Pept Res. July 1999; 54(1):23-31.

11. Isner and Asahara: Angiogenesis and vasculogenesis as therapeutic strategies for postnatal neovascularization. J Clin Invest. May 1999; 103(9):1231-6.

12. Kallianpur A R, Jordan J E, Brandt S J: The SCL/TAL-1 gene is expressed in progenitors of both the hematopoietic and vascular systems during embryogenesis. Blood 83:1200, 1994.

13. Koblizek T I, Runting A S, Stacker S A, Wilks A F, Risau W, Deutsch U: Tie2 receptor expression and phosphorylation in cultured cells and mouse tissues. Eur.J. Biochem. 244:774, 1997.

14. Molecular Cloning, eds. Sambrook, Fritsch, and Maniatis, (1989).

15. Raz A, Levine G, Khomiak Y. Acute local inflammation potentiates tumor growth in mice. Cancer Lett. Feb. 1, 2000; 148(2):1 15-20.

16. Remington's Pharmaceutical Sciences (Martin, E. W., ed., latest edition), Mack Publishing Co., Easton, Pa.

17. Rozdzinski E, Spellerberg B, van der Flier M, Bhattacharyya C, Hoepelman Al, Moran M A, Jarpe A, Putney S D, Starzyk R M, Tuomanen E. Peptide from a prokaryotic adhesin blocks leukocyte migration in vitro and in vivo. J Infect Dis. September 1995; 172(3):785-93.

18. Samaniego F, Markham P D, Gendelman R, Gallo R C, Ensoli B. Inflammatory cytokines induce endothelial cells to produce and release basic fibroblast growth factor and to promote Kaposi's sarcoma-like lesions in nude mice. J Immunol. Feb. 15, 1997;158(4):1887-94.

19. Schacht E. Osteoporosis in rheumatoid arthritis—significance of alfacalcidol in prevention and therapy. Z Rheumatol. 2000;59 Suppl 1:10-20.

20. Shalaby F, Rossant J, Yamaguchi T P, Gertsenstein M, Wu X-F, Breitman M L, Schuh A C: Failure of blood-island and vasculogenesis in Flk-1-deficient mice. Nature 376: 62, 1995.

21. Springer et al. VEGF gene delivery to muscle: potential role for vasculogenesis in adults. Mol Cell. November 1998; 2(5):549-58.

22. Langer, R. (1998) “Drug delivery and targeting” Nature 392:5-10.

23. Tam P. P, Behringer R R: Mouse gastrulation: the formation of a mammalian body plan. Mech. Dev. 68:3, 1997.

24. Weidner, et al., 1991, Tumor angiogenesis and metastasis—correlation in invasive breast carcinoma. N. Engl. J. Med. Jan. 3, 1991;324(1):1-8.

What is claimed is:

1. A method of screening for an agent that promotes vasculogenesis, comprising the steps of
(a) contacting one or more embryonic vascular networks with the agent to be screened, under conditions in which extraembryonic mesodermal stem cells, or derivatives thereof, can migrate to the embryonic vascular network or networks;
(b) detecting, in the vascular network or networks, endothelial cells or endothelial cell precursors derived from extraembryonic mesodermal stem cells, or derivatives thereof; and
(c) comparing the endothelial cells or endothelial cell precursors derived from extraembryonic mesodermal stem cells, or derivatives thereof, in the networks contacted with the agent to be screened, with the endothelial cells or endothelial cell precursors derived from extraembryonic mesodermal stem cells, or derivatives thereof, in untreated networks,
an increase in endothelial cells or endothelial cell precursors derived from extraembryonic mesodermal stem cells, or derivatives thereof, in the network or networks contacted with the agent to be screened indicating an agent that promotes vasculogenesis.

2. The method of claim 1, wherein endothelial cells or endothelial cell precursors are detected by one or more markers selected from the group consisting of TAL1, Flk1, CD34, VE-cadherin, Tie 2, and platelet/endothelial cell adhesion molecule (PECAM).

3. The method of claim 1, wherein extraembryonic stem cells comprise a detectable tag.

4. The method of claim 3, wherein the detectable tag is a fluorescent label.

5. The method of claim 1, wherein the contacting step is performed in the whole embryo.

6. The method of claim 1, wherein the contacting step is performed ex vivo.

7. The method of claim 1, wherein the extraembryonic mesodermal stem cells are derived from blood islands or on allantois.

8. A method of promoting vasculogenesis in a tissue or organ, comprising contacting the tissue or organ with an agent identified by the screening method of claim 1.

9. A method of screening for an agent that promotes vasculogenesis, comprising the steps of
(a) co-culturing extraembryonic mesodermal stem cells and intraembryonic mesodermal stem cells, under conditions that allow formation of one or more vascular networks;
(b) contacting the co-culture with the agent to be screened;
(c) detecting, in one or more vascular networks, endothelial cells or endothelial cell precursors derived from extraembryonic mesodermal stem cells, or derivatives thereof; and
(d) comparing the endothelial cells or endothelial cell precursors derived from extraembryonic mesodermal stem cells, or derivatives thereof, in the vascular network or networks in the culture contacted with the agent to be screened, with the endothelial cells or endothelial cell precursors derived from extraembryonic mesodermal stem cells, or derivatives thereof, in the vascular network or networks of the untreated cultures,
an increase in endothelial cells or endothelial cell precursors derived from extraembryonic mesodennal stem cells, or derivatives thereof, in the vascular networks in the culture contacted with the agent to be screened indicating an agent that promotes vasculogenesis.

10. The method of claim 9, wherein endothelial cells or endothelial cell precursors are detected by one or more markers selected from the group consisting of TAL1, Flk1, CD34, VE-cadherin, Tie 2, and platelet/endothelial cell adhesion molecule (PECAM).

11. The method of claim 9, wherein extraembryonic stem cells comprise a detectable tag.

12. The method of claim 11, wherein the detectable tag is a fluorescent label.

13. A method of promoting vasculogenesis in a tissue, organ, or tumor, comprising contacting the tissue, organ, or tumor with an agent identified by the screening method of claim 9.

14. A method of screening for an agent that inhibits vasculogenesis, comprising the steps of
(a) contacting one or more embryonic vascular networks with the agent to be screened, under conditions in which extraembryonic mesodermal stem cells, or derivatives thereof, can migrate to the embryonic vascular network or networks;
(b) detecting, in the vascular network or networks, endothelial cells or endothelial cell precursors derived from extraembryonic mesodermal stem cells, or derivatives thereof, and
(c) comparing the endothelial cells or endothelial cell precursors derived from extraembryonic mesodermal stem cells, or derivatives thereof, in the networks contacted with the agent to be screened, with the endothelial cells or endothelial cell precursors derived from extraembryonic mesodermal stem cells, or derivatives thereof, in untreated networks,
an decrease in endothelial cells or endothelial cell precursors derived from extraembryonic mesodermal stem cells, or derivatives thereof, in the network or networks contacted with the agent to be screened indicating an agent that inhibits vasculogenesis.

15. The method of claim 14, wherein endothelial cells or endothelial cell precursors are detected by one or more markers selected from the group consisting of TAL1, Flk1, CD34, VE-cadherin, Tie 2, and platelet/endothelial cell adhesion molecule (PECAM).

16. The method of claim 14, wherein extraembryonic stem cells comprise a detectable tag.

17. The method of claim 14, wherein the detectable tag is a fluorescent label.

18. The method of claim 14, wherein the contacting step is performed in the whole embryo.

19. The method of claim 14, wherein the contacting step is performed ex vivo.

20. The method of claim 14, wherein the extraembryonic mesodermal stem cells are derived from blood islands or allantois.

21. A method of inhibiting vasculogenesis in a tissue or organ, comprising contacting the tissue or organ with an agent identified by the screening method of claim 14.

22. A method of treating a vasculogenic-dependent disease in a subject, comprising administering to the subject an agent identified by the screening method of claim 14.

23. A method of screening for an agent that inhibits vasculogenesis, comprising the steps of
(a) co-culturing extraembryonic mesodermal stem cells and intraembryonic mesodermal stem cells, under conditions that allow formation of vascular networks;
(b) contacting the co-culture with the agent to be screened;
(c) detecting, in vascular networks, endothelial cells or endothelial cell precursors derived from extraembryonic mesodermal stem cells, or derivatives thereof; and
(d) comparing the endothelial cells or endothelial cell precursors derived from extraembryonic mesodermal stem cells, or derivatives thereof, in the vascular networks in the culture contacted with the agent to be screened, with the endothelial cells or endothelial cell precursors derived from extraembryonic mesodermal stem cells, or derivatives thereof, in the vascular networks of the untreated cultures,
an decrease in endothelial cells or endothelial cell precursors derived from extraembryonic mesodermal stem cells, or derivatives thereof, in the vascular networks in the culture contacted with the agent to be screened indicating an agent that inhibits vasculogenesis.

24. The method of claim 23, wherein endothelial cells or endothelial cell precursors are detected by one or more markers selected from the group consisting of TAL1, Flk1, CD34, VE-cadherin, Tie 2, and platelet/endothelial cell adhesion molecule (PECAM).

25. The method of claim 23, wherein extraembryonic stem cells comprise a detectable tag.

26. The method of claim 23, wherein the detectable tag is a fluorescent label.

27. A method of inhibiting vasculogenesis in a tissue, organ, or tumor, comprising contacting the tissue, organ, or tumor with an agent identified by the screening method of claim 23.

28. A method of treating a vasculogenic-dependent disease in a subject, comprising administering to the subject an agent identified by the screening method of claim 23.
